(19) United States
(12) Patent Application Publication
(10) Pub. No.: US 2014/0339413 A1
(43) Pub. Date: Nov. 20, 2014

(54) METHOD FOR DETERMINING THE CONTENT OF ORGANIC SILICON COMPOUNDS IN ANTHROPOGENIC AND/OR BIOGENIC GASES CONTAINING METHANE

(71) Applicants: Thomas WORTELMANN, (US); G.A.S. Gesellschaft fuer analytische Sensorsyste mbH, Dortmund (DE)

(72) Inventors: Thomas Wortelmann, Dortmund (DE); Stefanie Sielemann, Dortmund (DE)

(73) Assignees: Thomas WORTELMANN, Dortmund (DE); G.A.S. Gesellschaft fuer analytische Sensorsysteme mbH, Dortmund (DE)

(21) Appl. No.: 14/368,669

(22) PCT Filed: Dec. 27, 2012
(86) PCT No.: PCT/EP2012/076916

§ 371 Date: Jun. 25, 2014

(30) Foreign Application Priority Data
Dec. 28, 2011 (DE) 10 2011 057 096.9
Mar. 8, 2012 (DE) 10 2012 101 945.2

(51) Int. Cl. G01N 27/62; G01N 33/00; G01N 33/497

(52) U.S. Cl. CPC G01N 27/622; G01N 33/497; G01N 33/0004; G01N 2033/4975; G01N 2033/4977
USPC 250/281

(57) ABSTRACT

The aim of the invention is to provide a solution by means of which the content of organic silicon compounds in anthropogenic and/or biogenic gases containing methane can be determined in the simplest and most reliable manner possible using a method for determining the content of organic silicon compounds in anthropogenic and/or biogenic gases containing methane, such as biogases, sewer gases, and landfill gases. The aim is achieved by using ion mobility spectrometry as a measurement method, wherein the anthropogenic and/or biogenic gas containing methane is used as a measurement gas.

The invention relates to a method for the determination of the content of organic silicon compounds in anthropogenic and/or biogenic gases containing methane, such as biogases, sewage treatment gases, landfill gases.

In the use of biogenic or anthropogenic gases containing metals to generate energy, such as biogas, sewage treatment gas, or landfill gas, various problems occur in practice, which are particularly caused by the organic silicon compounds (siloxanes) contained in the gases.

Organic components of household and industrial waste deposited in a landfill, for example, are biologically decomposed over time by microorganisms. At the end of this biological decomposition chain are methane bacteria that convert the residual organic components to form methane and carbon dioxide, with the exclusion of oxygen (anaerobically). This gas mixture, which generally contains between 50 and 60% methane and 35 to 45% carbon dioxide, is referred to as landfill gas. Methane reinforces the greenhouse effect and is therefore damaging to climate if it escapes into the environment in uncontrolled manner. Furthermore, because of its easy flammability, it is explosive under certain conditions.

For this reason, landfill gas is drawn out of the landfilled waste, in targeted manner, by means of a gas collection system, and passed along to be used in gas engines for energy. This is ecologically and economically practical, because in this way, the methane from the waste, which is damaging to climate, is converted to energy (electricity) in climate-neutral manner.

A gas collection system generally consists essentially of a plurality of vertical gas wells that are installed into the landfilled waste in a grid pattern of 50 m, for example. Using these gas wells, the landfill gas that forms is passed along to gas engines for the production of electrical energy, by way of gas lines, under a partial vacuum that is generated in compressors. Before the gas reaches the engines, water is removed from the landfill gas at multiple locations.

In general, filters are installed in the gas system ahead of the gas engines, with which filters the landfill gas is purified, generally at first with a main filter and a second downstream filter that is also referred to as a police filter and is supposed to prevent breakthrough of the substances if the main filter becomes full.

These filters are generally activated charcoal filters that are suitable for filtering out silicon-organic contaminants from the gas. Such siloxanes are formed during the metabolization of detergents, cosmetics, other skin care products, silicone-based oils, waterproofing agents, water-resistant materials, shoe polishes, surface protectants, etc., for example, by bacteria present in the landfills and sewage treatment plants. At high concentrations of these siloxanes, damage to the gas engines occurs, because the siloxanes are deposited on the pistons and valves of the gas engines. When these incrustations are split off, the valves leak and burn through, so that the cylinder head must be replaced. This leads to a reduced useful lifetime of the system and thereby to economic loss. Furthermore, the maintenance costs and times of the gas engines increase. Furthermore, deposits in the catalytic converters and thermal reactions lead to the result that the engines no longer work effectively.

The aforementioned filters serve to prevent these harmful influences on the gas engines, but their ability to function must be guaranteed. Up to the present, it has been usual in practice, for this purpose, to take a gas sample from time to time and to have its siloxane content, in each instance, determined in a laboratory, whereupon it can then be determined whether or not filter replacement is necessary. However, such a measurement method is cost-intensive and time-consuming, and generally requires several days. In practice, this leads to the result that filters are changed too late, so that the gas engines can be damaged by the siloxanes, or that filters are changed prematurely, for preventive purposes, and this is connected with unnecessary additional costs.

There is therefore a need to be able to determine the siloxane content in such systems continuously or quasi-continuously, in order to be able to replace a filter at the proper time. For this purpose, real-time measurements by means of infrared siloxane analysis devices have already become known (e.g. JP 2008196870 A or JP2011033636A). However, these measurement methods have not established themselves in practice up to the present, because they are not sufficiently selective and are influenced by a changing moisture content of the gas.

It is the task of the invention to create a solution with which the content of inorganic silicon compounds in anthropogenic and/or biogenic gases containing methane can be determined in the most simple and reliable manner possible.

This task is accomplished, according to the invention, in a method of the type indicated initially, in that ion mobility spectrometry is used as the measurement method, wherein the anthropogenic and/or biogenic gas containing methane is used as the measurement gas.

Ion mobility spectrometers have fundamentally been known for quite some time for other purposes of use. Thus, such devices have already been designed and built for military use for decades, and are used, above all, as warning devices for chemical weapons or the like. Use of ion mobility spectrometry for detection and quantification of sulfur-free odorants in natural gas or fuel gas is also known (EP 1 499 881 B1).

It has now surprisingly been shown that ion mobility spectrometry is suitable for gases contaminated with siloxanes or organic silicon compounds, even under difficult conditions, and reliably allows continuous or discontinuous measurements of the siloxane content, in each instance. If predetermined limit values are exceeded, then depending on the type of system in which the gas in question is situated, a warning signal can be issued at first, for example, or, if necessary, the system can also be completely deactivated. In the case of landfill gas systems or biogas systems or sewage treatment gas systems, the effectiveness of the filters used can be monitored regularly, and required filter replacement can be determined at precisely the right point in time, to a great extent, so that filter breakthroughs are reliably prevented.

In a particularly preferred embodiment, it is provided that the anthropogenic and/or biogenic gas containing methane stands in continuous gas exchange, at least for the time of the analysis, with an ion mobility spectrometer in which the gas is ionized, and these ions are subsequently analyzed in a drift channel of the ion mobility spectrometer. Nitrogen, for example, can be used as a drift gas; it is easily available in such systems.

Alternatively, the method can also be carried out offline, in that a sample is taken from the gas and this sample is introduced into an ion mobility spectrometer as the measurement gas.

Depending on the configuration of the system, the ion mobility spectrometer can be embedded into the region of a system that is filled with an anthropogenic and/or biogenic gas containing methane, or can exist in exchange with the gas by way of a gas line. The gas line can be connected with the ion mobility spectrometer by way of a valve and a suction pump.

The method is particularly suitable for biogas systems, sewage treatment gas systems, or landfill gas systems.

If the ability of the gas filter or filters to function is to be monitored in such systems, it is preferably provided that the ion mobility spectrometer stands downstream from a gas filter of a gas engine of the system, in the gas exchange.

Furthermore, the ion mobility spectrometer or also a second ion mobility spectrometer can stand upstream from a gas filter of a gas engine of the system, in the gas exchange.

In order to reduce the measurement effort and thereby to obtain faster and more easily comprehensible measurement results, it is preferably provided that only the content of individual, pre-selected silicon compounds is determined. Typical siloxanes that occur in such systems are tetramethylsilane, trimethylsilanol, hexamethyldisiloxane, hexamethylcyclotrisiloxane, octamethylcyclotrisiloxane, octamethylcyclotetrasiloxane, decamethyltetrasiloxane, and decamethylcyclopentasiloxane. In order to achieve informative results, it is then sufficient, for example, to determine only the content of trimethylsilanol, octamethylcyclotrisiloxane, octamethylcyclotetrasiloxane, and decamethylcyclopentasiloxane.

The invention will be explained in greater detail below, using the drawing as an example. This shows, in

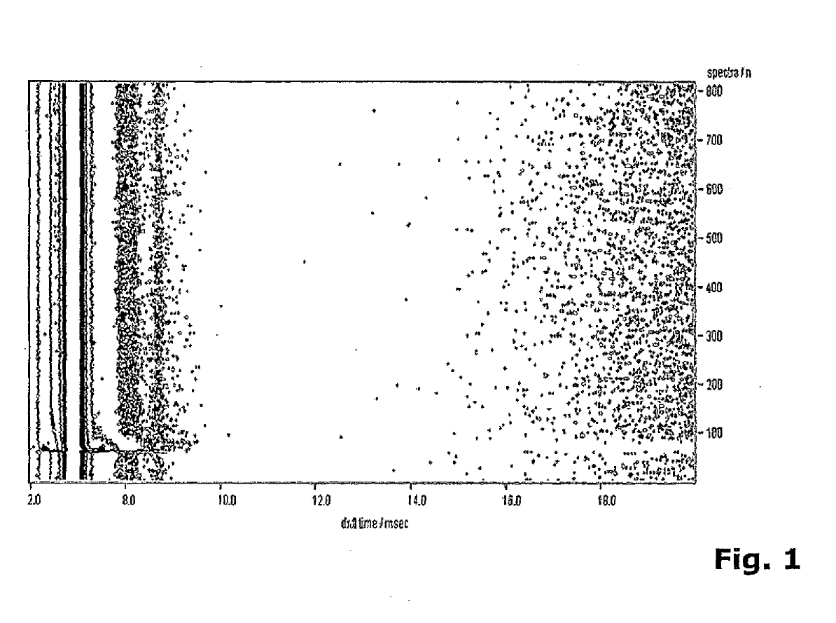

FIG. 1 an ion mobility spectrometer chromatogram of biogas without siloxane content, and in

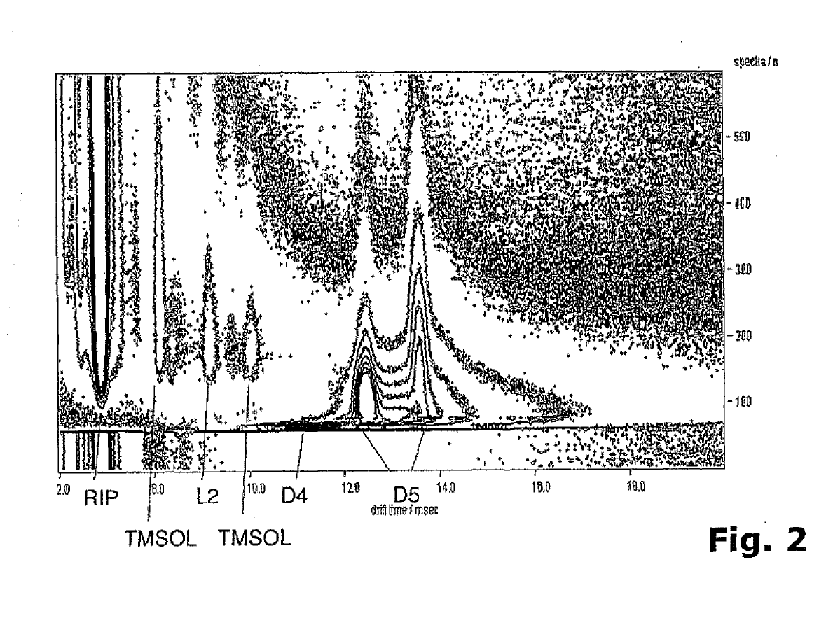

FIG. 2 an ion mobility spectrometer chromatogram of biogas with siloxane content, and in

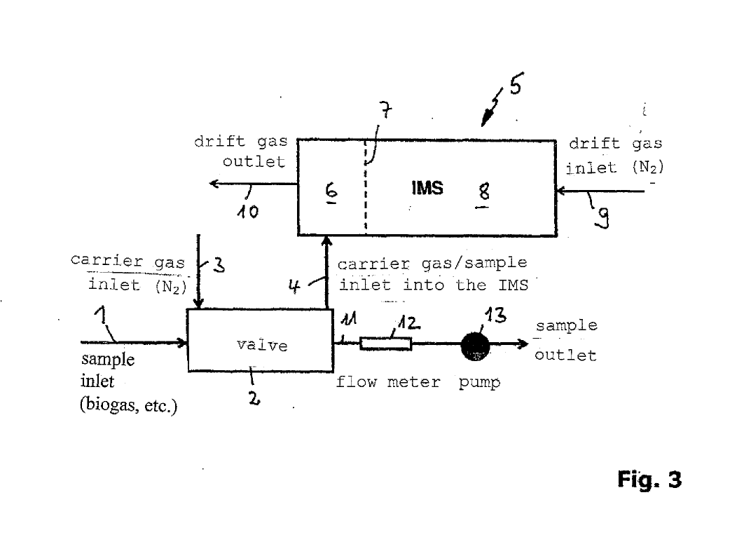

FIG. 3 a schematic representation of a measurement setup for carrying out the method according to the invention.

In FIG. 3, only a branch gas line 1 of a biogas system, landfill gas system or sewage treatment gas system is shown, which line stands in direct contact with the system downstream from a gas filter, directly ahead of a gas engine. The gas line 1 ends in a multi-way valve 2 that has multiple inlets and outlets; aside from the gas line 1, a carrier gas line 3 is provided as a further inlet. A carrier gas sample exit line 4 is provided as the outlet from the valve 2; this line opens into an ion mobility spectrometer indicated in general with 5. This ion mobility spectrometer 5 has the usual structure and is only indicated schematically; the line 4 opens into an ionization chamber 6 in which an ionization source, not shown, is disposed. The ionization chamber 6 is delimited by a switching grid 7 for ion swarm formation, followed by a drift chamber 8. On the end facing away from the grid 7, the drift chamber 8 has a collection electrode, not shown, for example in the form of a Faraday plate. In this region, a drift gas inlet 9, for example for nitrogen as the drift gas, is also provided, and, at the opposite end, a drift gas outlet 10 is provided. The valve 2 furthermore has a sample outlet line 11, in which a flow meter 12 and a pump 13 are disposed.

Because such biogas systems or landfill gas systems are usually operated at a slight partial vacuum, the pump 13 is required in order to feed the gas to be analyzed to the ion mobility spectrometer 5 by way of the gas line 1, and to analyze it in the spectrometer.

As has been shown to be true, ion mobility spectrometry is decidedly well suited for determining the content of organic silicon compounds in biogases, landfill gases, or sewage treatment gases.

In FIG. 1, the chromatogram of a typical biogas without siloxane content, i.e. a type of pure gas, is shown. In the left region, only what is called the reaction ion peak can be seen.

FIG. 2, in contrast, shows a measurement result of a gas charged with siloxanes. Aside from the usual reaction ion peak RIP, the substances tetramethylsilane (TMS), hexamethyldisiloxane (L2), octamethylcyclotetrasiloxane (D4), and decamethylcyclopentasiloxane (D5) can be clearly recognized.

Of course, the invention is not limited to the exemplary embodiments shown. Further embodiments are possible, without departing from the basic idea.

1. Method for the determination of the content of organic silicon compounds in anthropogenic and/or biogenic gases containing methane, such as biogases, sewage treatment gases, landfill gases,
wherein
ion mobility spectrometry is used as the measurement method, wherein the anthropogenic and/or biogenic gas containing methane is used as the measurement gas.

2. Method according to claim 1,
wherein
the anthropogenic and/or biogenic gas containing methane stands in continuous gas exchange, at least for the time of the analysis, with an ion mobility spectrometer in which the gas is ionized, and these ions are subsequently analyzed in a drift channel of the ion mobility spectrometer.

3. Method according to claim 1,
wherein
the ion mobility spectrometer is embedded into the region of a system that is filled with an anthropogenic and/or biogenic gas containing methane, or stands in exchange with the gas by way of a gas line.

4. Method according to claim 3,
wherein
the system is a biogas system, sewage treatment gas system, or landfill gas system.

5. Method according to claim 4,
wherein
the ion mobility spectrometer stands downstream from a gas filter of a gas engine of the system, in the gas exchange.

6. Method according to claim 4,
wherein
the ion mobility spectrometer stands upstream from a gas filter of a gas engine of the system, in the gas exchange.

7. Method according to claim 1,
wherein
only the content of individual, pre-selected silicon compounds is determined.
